(19) United States
(12) Patent Application Publication
(10) Pub. No.: US 2016/0143785 A1
(43) Pub. Date: May 26, 2016

(54) PROTECTIVE EYE MASK, PARTICULARLY FOR SKIING

(71) Applicant: BONDET LUNETTES, Oyonnax (FR)

(72) Inventors: CHRISTOPHE LAPERRIERE, ANNECY (FR); PIERRE BONDET, VEYZIAT (FR)

(21) Appl. No.: 14/889,174

(22) PCT Filed: Feb. 26, 2015
(86) PCT No.: PCT/IB2015/051419

§ 371 Date: Nov. 5, 2015

(30) Foreign Application Priority Data
Mar. 26, 2014 (FR) 14 52598

(51) Int. Cl. A61F 9/02

(52) U.S. Cl. CPC A61F 9/025

(57) ABSTRACT

The invention relates to a mask (1) including a basic structure (2) and at least one screen (3). The basic structure (2) has at least four through-holes (8) in the receiving surface of the screen (3), each hole having, along the length thereof, at least two enlargements spaced apart from one another, forming chambers having a larger cross-sections than the hole, one of the enlargements being located at a greater depth in the hole (8) and the other enlargement being located at a shallower depth in said hole. The screen (3) comprises at least four fingers (12) projecting from the inner surface thereof, which are arranged in alignment with the holes (8) and which comprise a widened portion (12a) suitable for insertion in either said enlargements. The insertion of all of said widened portions (12a) in all of said intermediate enlargements achieves a stable positioning of the screen (3) spaced apart from said receiving surface, such as to form a ventilation slit between the inner surface of the screen (3) and said receiving surface.

The present invention relates to a protective eye mask, in particular usable for sports, and in particular for skiing. This mask can be used not only for skiing, but also for motorcycle riding, or for any type of protection in general, in particular during home improvement work or for any industrial activity that requires protection for the eyes.

It is normal for a skier to protect his eyes from the reflection of the sun on the snow, the cold air or any snowflakes, using a mask comprising a base structure, and a screen mounted on the base structure. To prevent mist from forming on the screen due to the temperature difference between the inside and outside of the mask, it is known to arrange ventilation ducts through the base structure, which can be more or less open as needed using manipulable opening/closing means.

However, these masks are not fully satisfactory. In fact, the ventilation allowed by the aforementioned ducts is not perfect and does not preclude the possibility of the appearance of mist. Furthermore, the manipulation of said opening/closing members is not very quick or easy, and the skier is generally required to remove his gloves to perform that manipulation. As a result, the skier frequently chooses simply to place the mask on his forehead temporarily, such that his eyes are no longer protected.

Furthermore, a ski mask must allow easy switching between a dark screen, protecting from the reflection of the sun on the snow, and a yellow screen, improving vision in case of fog. On the existing masks, this switching is not always very quick or easy.

Published patent application No. US 2013/091623 A1 describes a mask usable for skiing, comprising a base structure and a screen that can be mounted on that base structure. The base structure has a surface bearing against a user's face and, opposite, a surface for receiving the screen, and comprises an upper portion, designed to extend against a user's forehead when the mask is worn, and a lower portion, designed to extend over the cheekbones and around the nose of the user when the mask is worn. The structure is made from an elastically deformable material; it has emerging holes in said receiving surface, at least two of which are arranged in said upper portion, and at least two others of which are arranged in said lower portion. Each hole has an inner widening, configured to allow snapping, and the screen includes at least four snapping teeth, with short lengths, protruding from its inner surface, i.e., its surface turned toward the user's face when the mask is worn; these teeth are positioned to match the holes comprised by the base structure and can be inserted and retained in the corresponding holes to mount the screen on the base structure.

Such a mask does not make it possible to resolve the aforementioned drawbacks.

Published patent applications Nos. US 2006/117468 A1, U.S. Pat. No. 5,018,223 A, U.S. Pat. No. 3,395,406 A, U.S. Pat. No. 4,653,124 A and GB 2,296,854 A describe similar masks, with a base structure comprising mounting holes and screens comprising snapping teeth, or other similar assembly parts, with shorter lengths, making it possible to assemble the screen to the base structure.

The present invention aims to resolve the aforementioned drawbacks.

Its main aim is therefore to provide a mask whereof the inner space can be ventilated easily when the mask is worn.

Another aim of the invention is to provide a mask that is quick and easy to manipulate, even with gloves.

Still another aim of the invention is to achieve at least one of the aforementioned aims with a mask whereof the structure remains relatively simple and easy to produce.

One additional aim of the invention is to provide a mask making it possible to achieve at least one of the aforementioned aims and further allowing quick and easy switching of one screen for another screen.

The mask comprises a base structure and at least one screen that can be mounted on that base structure; the base structure has a surface designed to bear against a user's face and, opposite, a surface receiving the screen, and comprises an upper portion, designed to extend against a user's forehead when the mask is worn, and a lower portion, designed to extend over the cheekbones and around the nose of the user when the mask is worn; the base structure is made from an elastically deformable material; the base structure has at least four holes emerging in said receiving surface, at least two of which are arranged in said upper portion and at least two others of which are arranged in said lower portion; each hole has a widening portion arranged below its opening; the screen has an inner face, turned toward the user's face when the mask is worn, and comprises at least four projections on this inner face, arranged in correspondence with the holes comprised by the base structure; each projection has a free end and has, on the side of this free end, a wider portion; each hole and each corresponding projection are configured to allow snapping of the projection in the hole, by receiving the wider portion comprised by the projection in said wider portion formed by said hole, allowing the projection to be kept in a stable engagement position, the engagement of the different projections in the different holes making it possible to mount the screen on said base structure;

wherein:

the aforementioned widening portions and wider portions are first widening portions and first wider portions, and said stable engagement position is a first stable engagement position;

each hole has an elongated shape allowing it to receive the corresponding projection in at least one second engagement position of that projection in that hole, offset relative to said first stable engagement position;

the corresponding projection has an elongated shape allowing it to be engaged in said hole in said first stable engagement position and said second engagement position, respectively; and

either said hole comprises a second widening portion separate from said first widening portion, or said projection comprises at least one second wider portion separate from said first wider portion, said second widening portion and/or said second wider portion allowing said second engagement position to be a stable engagement position.

The invention thus allows the existence of at least two stable engagement positions of the projections in the corresponding holes of the base structure; in one of these positions, the screen is pressed against said receiving surface, and in another of these positions, the screen is positioned stably away from said receiving surface so as to form a ventilation interstice between the inner surface of the screen and said receiving surface.

Thus, from the stable position in which the screen is kept pressed against said receiving surface, a pulling force exerted on the edge of the screen in the direction moving the screen away from that receiving surface makes it possible to move the wider portions of the projections in the holes to the stable position in which the screen is kept away from said receiving surface; the ventilation interstice thus arranged between the inner surface of the screen and that receiving surface allows effective ventilation of the volume delimited by the base structure and by the screen, in light of the significant length of that interstice and its presence on all, or a wide portion, of the perimeter of the screen. The screen is made easier to handle, and the pulling force exerted on its edge may be exerted without the user having to remove his gloves.

It must be noted that the flexibility of the base structure and/or said projections allows the upper part of the screen to be able to be placed away from the upper part of the base structure when the lower part of the screen remains pressed against the lower part of the structure, or vice versa, which allows two additional ventilation positions, in which the screen is inclined relative to the structure.

Preferably, said projections are all able to be removed from the corresponding holes, such that a pulling force exerted on the screen makes it possible to remove all of said wider portions of the projections outside all of the corresponding holes, which allows a separation of the screen and the base structure, for quick and easy switching of the screen. A replacement screen can thus be mounted equally quickly and easily.

In order to return the screen from the ventilation position to the position pressed against the base structure, it suffices to exert a thrust force on the screen so as to return all of the wider portions of the projections into all of the widening portions of the holes.

It will be understood that the “elongated shape” of each hole and each projection is intended to define the fact that the length of each hole and each projection is significantly larger than a transverse dimension (diameter or diagonal) of the hole and of that projection, i.e., at least three times that transverse dimension, typically three to five times that transverse dimension.

It will also be understood that the invention encompasses:

an embodiment in which each projection comprises a single wider portion and each hole comprises several widening portions;

an embodiment in which each hole comprises a single widening portion and each projection comprises several wider portions; and

an embodiment in which each hole comprises several widening portions and each projection comprises several wider portions.

Preferably, said base structure comprises at least one hole as cited above in each of its side portions connecting said upper portion to said lower portion, and the screen comprises at least one projection as cited above at each of its side portions, arranged in correspondence to said hole.

The screen is thus assembled at these side portions of the base structure and the screen.

According to one possibility, each aforementioned wider portion has a spherical shape and each widening portion has a corresponding spherical shape.

It is thus easier for the wider portions to go from one position to the other.

According to another possibility, each aforementioned hole is laterally delimited by two snapping teeth that are part of the base structure, those two snapping teeth being able to snap behind said first wider portion of the corresponding proJection and, if applicable, behind said second wider portion of the projection, to ensure that said projection is kept in that hole by snapping.

These snapping teeth can be formed by elastically deformable inserts embedded in the material making up the majority of the base structure at said holes.

These inserts make it possible to give the base structure the appropriate degree of elasticity to obtain the desired snapping.

Preferably, at least two consecutive projections are connected to one another by a junction strip fastened to the screen.

This junction strip provides a wide junction surface of the projections with respect to the screen, which allows strong fastening of those projections to that screen.

Advantageously, all of the projections are connected to one another by said junction strip, which then forms a continuous band over the perimeter of the screen.

The projections may in particular be integrally molded with that junction strip, and the projections-junction band assembly may be overmolded on the screen.

In order to ensure a perfect assembly of the screen with respect to the base structure, the latter may have four holes at its upper portion, four holes at its lower portion and one hole in each of its side portions, the screen consequently comprising ten corresponding projections.

According to one possibility, the screen is sized such that its contour edge protrudes beyond the outer contour of said receiving surface. The part of the screen protruding past that outer contour forms an engagement surface bearing against the screen, allowing easy manipulation of the latter.

According to another possibility, the screen is sized such that its contour edge fits inside the outer contour of said receiving surface, and the base structure has recesses at its upper and/or lower portions, allowing bearing engagement against the edge of the screen.

Preferably, said base structure comprises at least one mobile fastener allowing, in a locking position, blocking of the snapping teeth of a hole in a position engaged with the wider portion(s) of the corresponding projection and, in a release position, releasing the movement of the snapping teeth so as to allow the movement of that projection inside the hole.

This fastener thus makes it possible to lock the screen in one or the other of the aforementioned positions.

The invention will be well understood, and other features and advantages thereof will appear, in reference to the appended diagrammatic drawing, showing, as non-limiting examples, three possible embodiments of the mask in question.

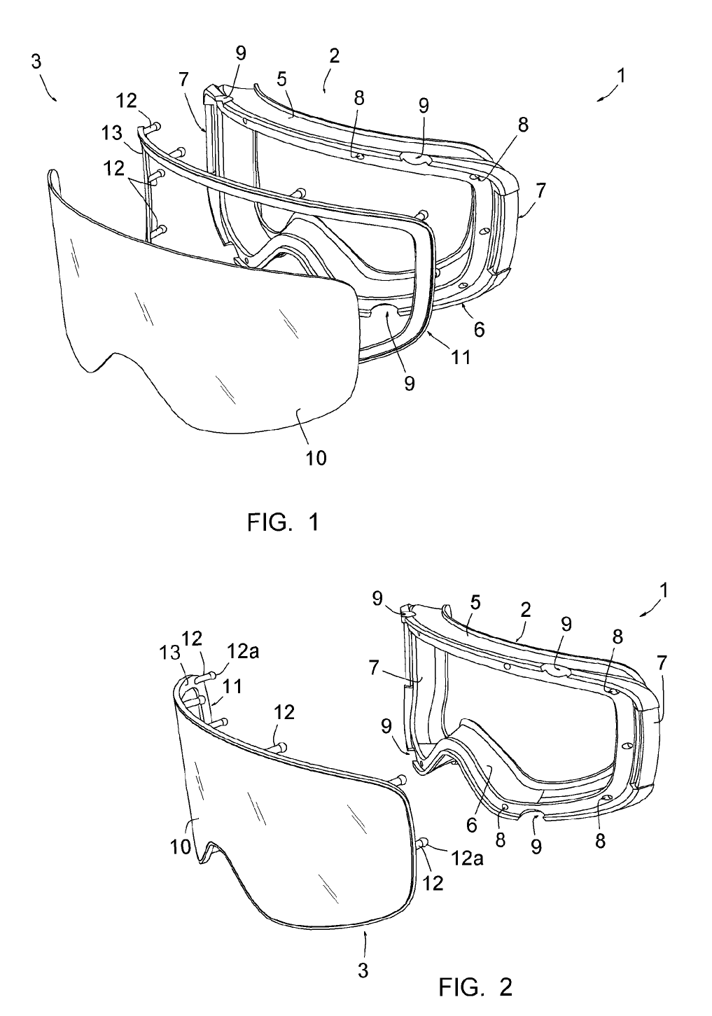

FIG. 1 is a view of that mask according to a first embodiment, in an exploded perspective view;

FIG. 2 is a perspective view, before assembly, of a screen that it comprises on a base structure that it also comprises;

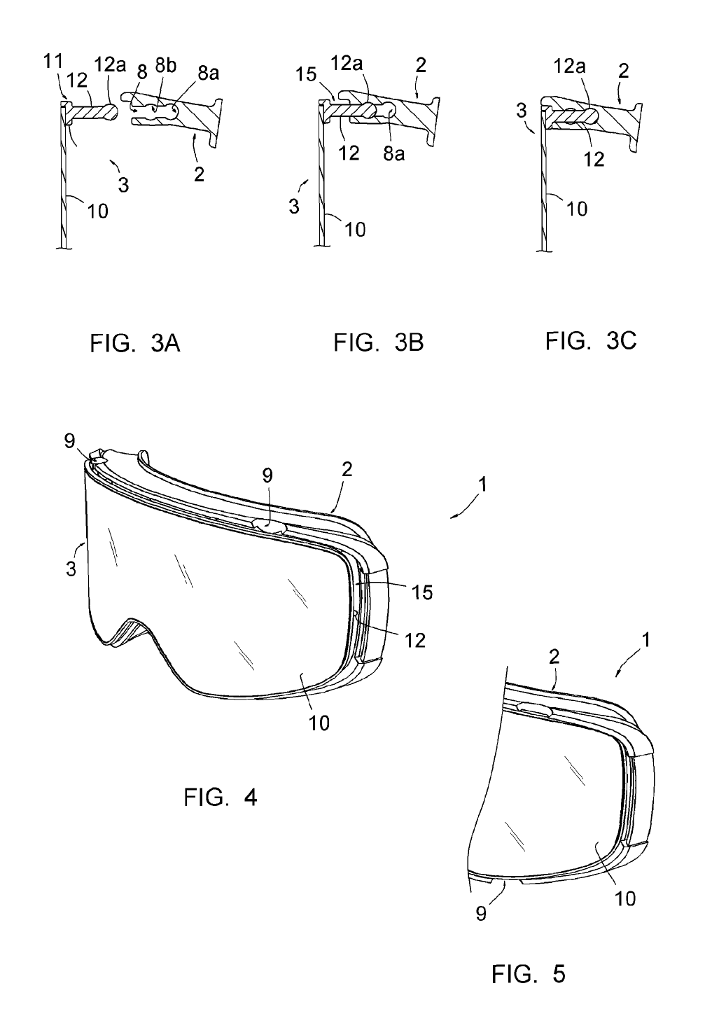

FIGS. 3A to 3C are sectional views of said screen and said base structure at the axis of one of the fingers comprised by the screen and the axis of the corresponding hole comprised by the base structure, in three different positions, namely, respectively, a disassembled position of the screen with respect to the base structure, a ventilation position in which the screen is mounted on the base structure but separated therefrom, and a position in which the screen is pressed against the base structure;

FIG. 4 is a view of mask similar to FIG. 2, in which the screen is in said ventilation position;

FIG. 5 is a partial view of the mask, similar to FIG. 4, in which the screen is in said position in which it is pressed against the base structure;

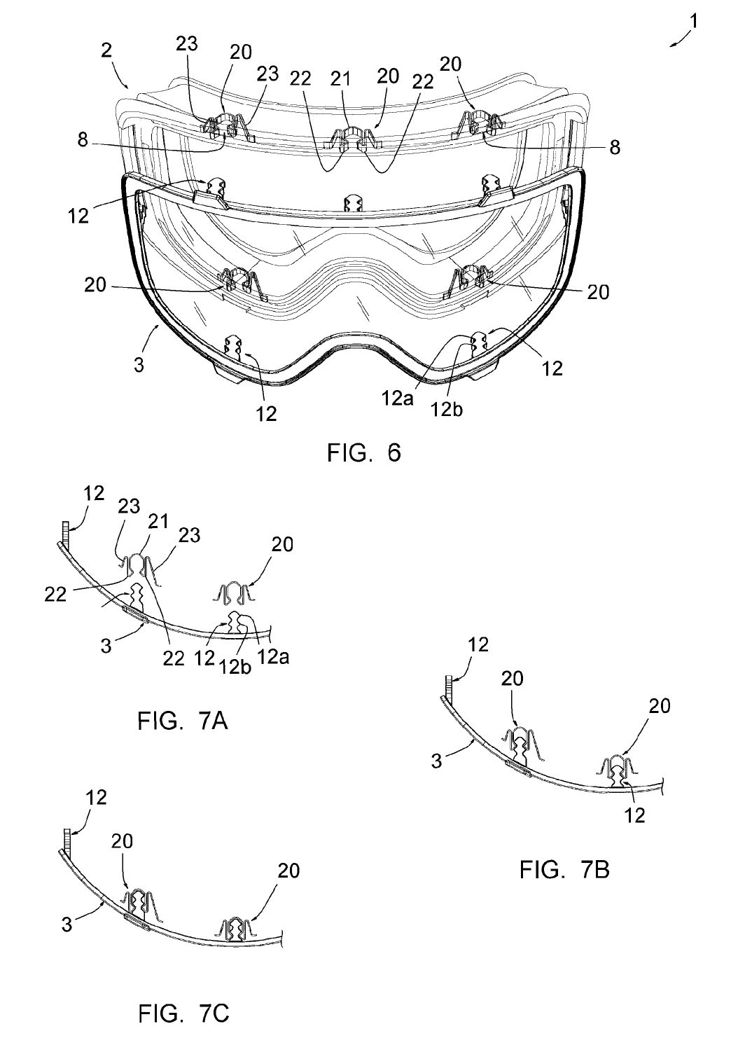

FIG. 6 is a view of the mask according to a second embodiment, in perspective view, before assembly of the screen comprised by that mask on the base structure that it also comprises;

FIGS. 7A to 7C are partial top views of the screen and inserts comprised by that base structure, in the same three respective positions as those shown in FIGS. 3A to 3C;

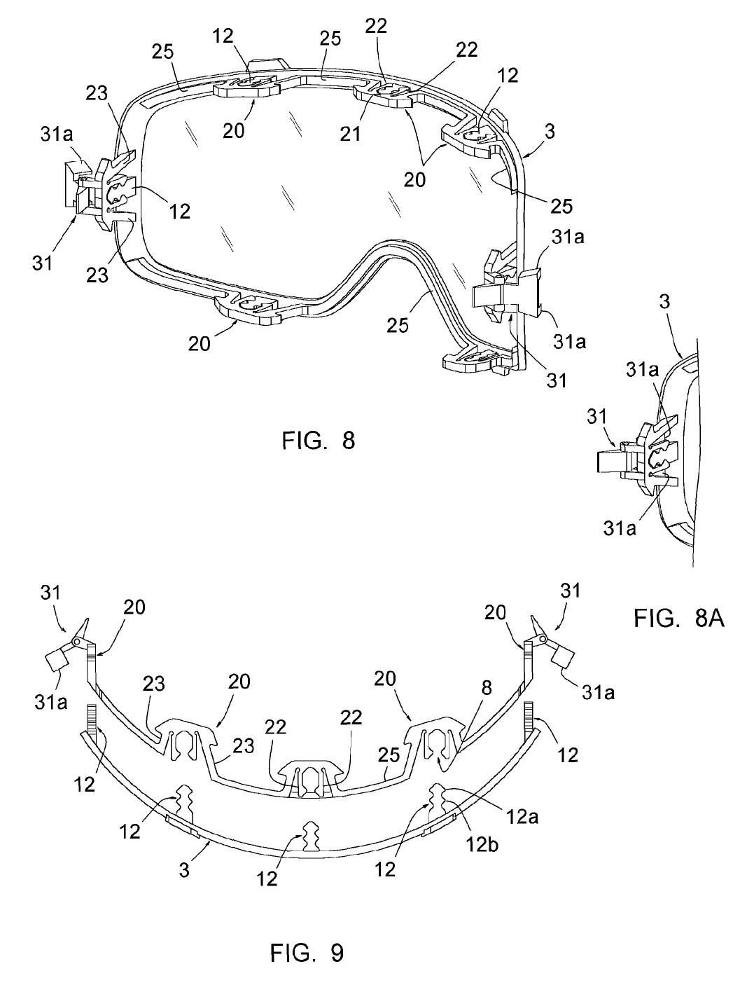

FIG. 8 is a perspective view of part of the base structure and the screen comprised by the mask according to a third embodiment, with assembly of the screen on the base structure; in this FIG. 8, side fasteners comprised by the mask are in a released position;

FIG. 8A is a partial view of the left side of the base structure and screen as shown in FIG. 8, on which the side fastener comprised by the mask is in a locking position; and

FIG. 9 is a top view of the screen and the base structure comprised by that mask, in the disassembled position of the screen with respect to the base structure.

FIGS. 1 and 2 show a protective mask 1 for the eyes, in particular usable for sports, and in particular skiing, that comprises a base structure 2 and a screen 3 that can be assembled on that base structure 2.

The base structure 2 has a curved shape adapted to the anatomy of a face. It is formed by a single molded part made from a slightly elastically deformable synthetic material, identical to those generally used to produce such ski mask base structures.

The base structure 2 has a surface adapted to bear against a user's face and, opposite, a surface for receiving the screen 3; in the illustrated example, this receiving surface is delimited by a peripheral wall adjusted to the outer contour of the screen 3, as in particular shown in FIGS. 3A to 3C.

Said bearing surface includes a foam adapted for a gentle contact with the user's face, not shown in these FIGS. 3A to 3C.

The base structure 2 comprises an upper portion 5, designed to extend against a user's forehead when the mask 1 is worn, a lower portion 6, designed to extend over the cheekbones and around the nose of the user, and side portions 7 connecting those portions 5 and 6. The base structure 2 has ten holes 8 with parallel axes, identical to one another, emerging in said receiving surface and extending in the thickness of the base structure 2. Four holes are situated at its upper portion 5, four others at its lower portion 6 and one in each of its side portions 7. FIG. 2 in particular shows that the holes 8 are fairly regularly distributed on the perimeter of said receiving surface, with the two upper side holes 8 situated near or at the ends of said upper portion 5, the two lower side holes 8 situated near the ends of said lower portion 6 and the two holes 8 of the side portions 7 situated substantially at the center of those side portions.

The axes of said holes 8 are perpendicular to a plane containing points of the base structure 2 that are symmetrical relative to the median portion of that base structure 2.

In reference to FIGS. 3A to 3C, it appears that each hole 8 has an elongated shape, according to which the length of that hole is approximately four times its diameter, and the hole 8 comprises, over its length, two spherical widening portions 8a, 8b separated from one another, forming chambers with a larger section than the diameter of the hole 8 in other portions of the hole. The portions of the hole 8 situated outside these widening portions 8a, 8b comparatively form constrictions. One of the widening portions 8a is situated at the bottom of the hole 8, therefore at a greater depth in that hole, and will be called “deep” below so as to distinguish it from the other widening portion; that other widening portion 8b is situated at a shallower depth in the hole 8, substantially at mid-depth of the hole in the illustrated example, and will be called “intermediate” below.

The base structure 2 also has two recesses 9 at its upper portion 5 and two identical recesses 9 at its lower portion 6, which extend at said receiving surface and allow bearing engagement against the edge of a part 10 comprised by the screen 3, as will appear below.

The side portions 7 comprise assembly loops for an elastic strap (not shown), well known in themselves and therefore not described in more detail.

The screen 3 is formed by a transparent part 10, making up the screen strictly speaking for protecting the user's eyes, and by a junction part 11 joining the part 10 to the base structure 2.

The part 10 has a curved configuration, suitable for following the contour of the receiving surface of the base structure 2.

The part 11 comprises ten protruding fingers 12 and a base part 13 fastened to the part 10. These fingers 12 and base part 13 are formed by a single piece of molded synthetic material, in particular overmolded on the part 10.

The fingers 12 protrude from the base part 13 along axes parallel to one another; similarly to the axes of the holes 8; these axes of the fingers 12 are perpendicular to a plane containing points of the part 10 that are symmetrical relative to the median portion of that part 10. The fingers 12 are identical to one another and have a distribution identical to that of the holes 8; they are therefore positioned matching these holes 8 when the screen 3 is placed across from the base structure and are designed to be inserted into said holes to assemble the screen 3 on the base structure 2.

As particularly shown in FIGS. 3A to 3C, each finger 12 has an elongated shape, according to which its length is approximately four times its diameter. Each finger 12 comprises a terminal spherical portion 12a, wider relative to the rest of the finger, that rest of the finger forming a portion that is narrower compared to the widening portion formed by said terminal spherical portion 12a. This terminal spherical portion 12a has a diameter substantially corresponding to that of the widening portions 8a, 8b of the corresponding hole 8, and the rest of the finger 12 has a diameter substantially corresponding to the diameter of that corresponding hole 8 out of the widening portions 8a, 8b.

The base part 13 connects all of the fingers 12 to one another and therefore forms a continuous band extending over the perimeter of the part 10. It is fastened to the latter such that once that fastening is done, the fingers 12 protrude from the inner surface of that part 10.

As will be understood in reference to FIGS. 3A to 5, from the position of the screen 3 pressed against said receiving surface, shown in FIGS. 3C and 5, a pulling force is exerted on the edge of the screen 3 in the direction separating the screen from said receiving surface, said pulling force being done by bearing of the user's fingers against the screen 3 at the recesses 9. This pulling force makes it possible to place all of said wider portions 12a in said intermediate widening portions 8b, and therefore to position the screen 3 away from said receiving surface, said screen being kept stably in that position; the ventilation interstice 15 thus arranged between the inner surface of the screen 3 and the receiving surface (cf. FIGS. 3B and 4) allows effective ventilation of the volume delimited by the base structure 2 and the screen 3, in light of the significant length of that interstice 15 and its presence on the assembly, or a wide portion, of the perimeter of the screen 3. This position of the screen is stable due to the retention of said wider portions 12a in said intermediate widening portions 8b, by elastic return of the material making up the base structure 2. The manipulation of the screen 3 is easy, and the pulling force exerted on its edge may be exerted without the user having to remove his gloves.

It bears note that the flexibility of the base structure 2 makes it possible for the wider portions 12a of the fingers 12 situated in the upper portion of the screen 3 to be able to be brought into the intermediate widening portions 8b of the corresponding holes, while the wider portions 12a of the fingers situated on the lower portion of the screen 3 are kept in said deep widening portions 8a of the corresponding holes, or vice versa, which allows two additional ventilation positions, in which the screen 3 is inclined relative to the base structure 2.

An additional pulling force exerted on the screen 3 makes it possible to remove all of said wider portions 12a from all of the corresponding holes 8, which allows a quick and easy separation of the screen 3 and the base structure 2, in order to switch the screen 3. The assembly of the replacement screen is done equally quickly and easily.

In order to return the screen from the ventilation position to said pressed position, it suffices to exert a thrust force on the screen 3 so as to return all of said wider portions 12a into all of said deep widening portions 8a.

FIGS. 6 to 7C show a second embodiment of the mask 1; for simplification reasons, the parts or elements already described and that are also found in this second embodiment will be designated using the same numerical references and will not be described again.

In this second embodiment, the base structure 2 (shown in silhouette in FIG. 6 and fictitiously shown as being transparent) includes inserts 20 embedded in the material that essentially makes up the base structure 2. Each insert 20 is situated at a hole 8 and is formed by an elastic metal bar. This bar forms:

a round central part 21,

two snapping teeth 22 bordering said central part 21 and delimiting therewith an elongated widening portion of the hole 8; the teeth 22 form tips opposite one another, which jointly form a constriction of the hole 8 near the opening of the hole; and

two outer side parts 23 allowing anchoring of the insert 20 to the material making up the majority of the base structure 2.

Each finger 12 has a flat shape and has, aside from the terminal wider portion 12a, a second wider portion 12b separated from the portion 12a by a narrower portion. As will be understood in reference to FIGS. 7A to 7C, these wider portions 12a, 12b can be inserted into the widening portion of the hole 8 delimited by said central part 21 and the teeth 22, and the narrower portions separating those wider portions 12a, 12b are able to cooperate with the tips of the teeth 22 so as to allow the aforementioned three positions of the screen 3 relative to the base structure 2, i.e., said disassembly position (FIG. 7A), ventilation position (FIG. 7B) and position pressed against the base structure 2 (FIG. 7C).

FIGS. 8, 8C and 9 show a third embodiment of the mask 1, similar to the second embodiment described above, in which the inserts 20 are made from a synthetic material and connected to one another by junction portions 25, said inserts 20 and portions 25 being coated by the synthetic material making up the majority of the base structure 2 (that material is not shown). The portions 25 can extend over only part of the perimeter of the base structure 2, being interrupted at the side portions of the latter as shown in FIG. 8, or may extend over all of that perimeter.

In each insert 20, the central part 21, the snapping teeth 22 and the outer side parts 23 are molded in a single piece. Each insert 20, when it is connected to portions 25, also forms a single piece with those portions 25.

The mask 1 according to this third embodiment is used in the same way as that according to the second embodiment, as shown in FIGS. 7A to 7C.

This mask 1 further comprises side fasteners 31 mounted pivoting on two inserts 20 situated in a lateral position. Each fastener 31 comprises a U-shaped engaging head, the side walls 31a of which are configured and dimensioned so as to be able to be inserted into the existing spaces, in the corresponding insert 20, between the teeth 22 and said outer side parts 23, see FIG. 8A. These fasteners 31 thus make it possible to lock the teeth 22 in the engaged position with the wider portions of the corresponding fingers 12 and effectively keep those teeth 22 engaged with those wider portions.

In their released positions shown in FIGS. 8 and 9, these fasteners 31 release the movement of the teeth 22 so as to allow the movement of the fingers 12 inside the holes 8.

As appears from the preceding, the invention provides a protective mask for the eyes, in particular usable for sports, and in particular skiing, with decisive advantages, described above.

The invention has been described above in reference to embodiments provided as examples. It is of course not limited to these embodiments, but encompasses all other embodiments covered by the appended claims.

1. Protective eye mask (1), in particular usable for sports, and in particular for skiing, comprising a base structure (2) and at least one screen (3) that can be mounted on that base structure (2); the base structure (2) has a surface designed to bear against a user's face and, opposite, a surface receiving the screen (3), and comprises an upper portion (5), designed to extend against a user's forehead when the mask (1) is worn, and a lower portion (6), designed to extend over the cheekbones and around the nose of the user when the mask (1) is worn; the base structure (2) is made from an elastically deformable material; the base structure (2) has at least four holes (8) emerging in said receiving surface, at least two of which are arranged in said upper portion (5) and at least two others of which are arranged in said lower portion (6); each hole (8) has a widening portion (8a) arranged below its opening; the screen (3) has, on its inner face, i.e. its face turned toward the user's face when the mask (1) is worn, at least four projections (12), arranges in correspondence to the holes (8) comprised by the base structure (2); each projection (12) has, on the side of its free end, a wider portion (12a); each hole (8) and each corresponding projection (12) are configured to allow snapping of the projection (12) in the hole, by receiving the wider portion (12a) comprised by the projection (12) in said wider portion (12a) formed by said hole (8), allowing the projection (12) to be kept in a stable engagement position, the engagement of the different projections (12) in the different holes (8) making it possible to mount the screen (3) on said base structure (2);
characterized in that:
the aforementioned widening portions (8a) and wider portions (12a) are first widening portions (8a) and first wider portions (12a), and said stable engagement position is a first stable engagement position;
each hole (8) has an elongated shape allowing it to receive the corresponding projection (12) in at least one second engagement position of that projection (12) in that hole, offset relative to said first stable engagement position;
the corresponding projection (12) has an elongated shape allowing it to be engaged in said hole (8) in said first stable engagement position and said second engagement position, respectively; and
either said hole (8) comprises a second widening portion (8b) separate from said first widening portion (8a), or said projection (12) comprises at least one second wider portion (12b) separate from said first wider portion (12a), said second widening portion (8b) and/or said second wider portion (12b) allowing said second engagement position to be a stable engagement position.

2. The protective eye mask (1) according to claim 1, characterized in that said projections (12) are all able to be removed from the corresponding holes (8), such that a pulling force exerted on the screen (3) makes it possible to remove all of said wider portions (12a, 12b) of the projections (12) outside all of the corresponding holes (8), which allows a separation of the screen (3) and the base structure (2).

3. The protective eye mask (1) according to claim 1, characterized in that each projection (12) comprises a single wider portion (12a) and each hole (8) comprises several widening portions (8a, 8b).

4. The protective eye mask (1) according to claim 1, characterized in that each hole (8) comprises a single widening portion (8c) and in that each projection (12) comprises several wider portions (12a, 12b).

5. The protective eye mask (1) according to claim 1, characterized in that each hole comprises several widening portions and each projection comprises several wider portions.

6. The protective eye mask (1) according to claim 1, characterized in that said base structure (2) comprises at least one hole (8) in each of its side portions (7) connecting said upper portion (5) to said lower portion (6), and in that the screen (3) comprises at least one projection (12) at each of its side portions, arranged in correspondence to said hole.

7. The protective eye mask (1) according to claim 1, characterized in that each wider portion (12a) has a spherical shape and each widening portion (8a, 8b) has a corresponding spherical shape.

8. The protective eye mask (1) according to claim 1, characterized in that each hole (8) is laterally delimited by two snapping teeth (22) that are part of the base structure (2), those two snapping teeth being able to snap behind said first wider portion (12a) of the corresponding projection (8) and, if applicable, behind said second wider portion (12b) of this projection (12), to ensure that said projection (12) is kept in that hole (8) by snapping.

9. The protective eye mask (1) according to claim 8, characterized in that these snapping teeth (22) are formed by elastically deformable inserts (20) embedded in the material making up the majority of the base structure (2) at said holes (8).

10. The protective eye mask (1) according to claim 8, characterized in that at least two consecutive projections (12) are connected to one another by a junction strip (13) fastened to the screen (3).

11. The protective eye mask (1) according to claim 10, characterized in that all of the projections (12) are connected to one another by said junction strip (13), which forms a continuous band over the perimeter of the screen (3).

12. The protective eye mask (1) according to claim 1, characterized in that the base structure (2) have four holes (8) at its upper portion (5), four holes (8) at its lower portion (6) and one hole (8) in each of its side portions (7), the screen (3) consequently comprising ten corresponding projections (12).

13. The protective eye mask (1) according to claim 1, characterized in that the screen (3) is sized such that its contour edge protrudes beyond the outer contour of said receiving surface.

14. The protective eye mask (1) according to claim 1, characterized in that the screen (3) is sized such that its contour edge fits inside the outer contour of said receiving surface, and in that the base structure (2) has recesses (9) at its upper and/or lower portions (6), allowing bearing engagement against the edge of the screen (3).

15. The protective eye mask (1) according to claim 1, characterized in that said base structure (2) comprises at least one mobile fastener allowing, in a locking position, blocking of the snapping teeth (22) of a hole (8) in a position engaged with the wider portion(s) (12a, 12b) of the corresponding projection (12) and, in a release position, releasing the movement of the snapping teeth (22) so as to allow the movement of that projection (12) inside the hole.
